(19) United States
(12) Patent Application Publication
(10) Pub. No.: US 2012/0189553 A1
(43) Pub. Date: Jul. 26, 2012

(54) Dental cleaning composition for evidencing dental plaque

(76) Inventor: Lawrence A. Hier, Boca Raton, FL (US)

(21) Appl. No.: 13/013,441

(22) Filed: Jan. 25, 2011

(51) Int. Cl. A61K 9/00

(52) U.S. Cl. 424/9.71

(57) ABSTRACT

The present invention relates to oral compositions for identifying plaque comprising a dental disclosing agent composition for revealing the presence of dental plaque in a person's mouth. The dental disclosing agent composition includes natural colorants derived from a red beet extract, a pomegranate concentrate, and one or more of lycopene, red beet powder extract, and purple carrot extract powder, and a dental disclosing agent delivery composition for delivering the dental disclosing agent to a person's mouth.

### FIELD OF THE INVENTION

This invention relates generally to dental cleaning compositions for evidencing dental plaque, and in particular to a composition having natural colorants extracted from fruits or vegetables which can be used in orthodontic and non-orthodontic users.

### BACKGROUND ART

Our society has allowed unhealthy food and drinking products to be marketed directly toward children, as evident by the current high obesity rate in children. In addition, many of the products have ingredients that can promote tooth decay. For instance, refined sugar used in a variety of junk foods and soda drinks leads to tooth decay. In such an instance the plaque acids demineralize the teeth, beginning the tooth decay process. The process usually starts within a few minutes after the ingestion of refined sugar. The sugar laced products can further cause saliva to become acidic causing tooth decay and periodontal disease. See Glinsmann, et al. “Evaluation of Health Aspects of Sugar Contained in Carbohydrate Sweeteners” F.D.A. Report of Sugars Task Force—1986.

While tooth decay is an issue for everybody, the amount of decalcification and tooth decay found in orthodontic patients is alarming. Orthodontic devices are used to address a number of malocclusions including tooth irregularity, disproportionate jaw relationships, or both. For instance, braces are widely used orthodontic devices for correcting of crooked teeth, underbites, overbites, cross bite and open bites, widening the palate or jaws, or correcting other flaws of the teeth and jaws. At present, it is estimated that approximately one in every two patients has enamel decalcification upon removal of their fixed orthodontic devices. While such dental issues are described primarily using orthodontic patients as an example, the instant invention is not limited such users

The problem mentioned above applies to all patients or users, including those having orthodontic implants or those that do not, and can be rectified by proper diet, teeth cleaning after each meal, and abstinence from eating between meals. Unfortunately, teeth cleaning is more than simply a quick use of a tooth brush after a meal, proper teeth cleaning is an exercise that must be understood to be effective. The problems in the cleaning of teeth is further complicated when an orthodontic device, such as braces, are installed. It should be noted that the majority of orthodontic devices in use are placed on children and teenagers.

Even with proper oral hygiene instruction, young patients simply do not fully grasp the future ramifications that will result from improper teeth cleaning and poor oral hygiene. Thus, a child or teenager may brush their teeth after each meal as they have been taught throughout the years. However, due to the orthodontic device installed, they fail to actually clean their teeth properly. Thus, plaque acids that are concealed by the braces will continue to demineralize the teeth, leading to tooth decay. However if the patient knew where the problem existed (unclean area of teeth exist), even a child could address the problem.

Numerous studies have been conducted showing the severity of this problem in the patient population. Each year, half of the three to five million patients who get braces in the United States suffer from early tooth decay. At present, approximately one in every two patients has enamel decalcification upon removal of their fixed orthodontic appliances. Despite proper oral hygiene instruction by the orthodontist, or general dentist, and staff, as well as the presentation to the patient of various dental care aids, this level remains unchanged.

An article by Sukhia entitled Enamel Decalcification in Orthodontic Patients (Pakistan Oral & Dental Journal Vol. 28, No. 2) describes enamel decalcification as one of the most common complications of post-orthodontic treatment. Lack of oral hygiene or dental neglect is the major culprit, causing both esthetic and psychological problems for the patient. Mild post-orthodontic decalcification demonstrates a clinical color change from white to yellowish-white stains. This loss of enamel can range from minor ‘white spot lesions’ to actual cavitations. Moderate post-orthodontic decalcification is characterized by yellowish-brown staining and surface roughness. In some cases, severe post-orthodontic enamel decalcification causes darker, yellowish-brown stains with ultimate loss of enamel. Furthermore, excessive consumption of chemically erosive foods such as citrus fruits and fizzy carbonated drinks may aggravate any existing decalcification during treatment. In spite of various treatment options available today such as topical fluoride gels, toothpastes, mouthwashes, varnishes, acid-pumice micro abrasion and esthetic restorations, enamel decalcification remains a major unpredictable complication.

An article by Mattousch published on May 5, 2007 entitled “Caries lesions after orthodontic treatment followed by quantitative light-induced fluorescence” showed that the generally expected improvement of white spot lesions after the removal of fixed appliances and restoration of oral hygiene does not apply. Of the study, sixteen lesions were so severe that they required restoration. Only 10 of the 370 lesions remineralized completely after 2 years, while the majority of lesions found after orthodontic treatment were considered stable after 2 years of retention. These findings are in contrast with the general belief that lesions regress once the appliance is removed and oral hygiene is restored. Given the high number of lesions found at debonding in this study population, investigations focusing on remineralizing strategies for these types of lesions are necessary together with ongoing research to find more efficacious prevention treatments that can be used during fixed appliance treatment.

According to an article by Shannon et al entitled “Prevention of decalcification in orthodontic patients by daily self-treatment with 0.4% SnF gel” (Pediatric Dentistry Vol. 1, No. 2 1979), patients who used a gel daily remained completely free of decalcification. This makes it apparent that a good cleaning will prevent the aforementioned decalcification. In reality, no customer would maintain a gel on their teeth at all times.

In addition, the minimization of the microbial population in the mouth and the removal/prevention of plaque and calculus deposition are important for the prevention of tooth decay. Since the oral environment is conducive to microbial growth and subject to the reintroduction of food and microorganisms, and because plaque and calculus are continually being deposited on teeth, an ideal oral hygiene composition must be capable of cleansing and plaque removal, and be able to address the microbial growth. This situation is further complicated when the teeth are covered with braces and calls for treatment of the microbial problem during the cleaning process. Dental plaque which forms on tooth surfaces and restorations are colonies of harmful bacteria, which cannot be flushed away by simply rinsing with water. Active brushing of the teeth is required to remove said adherent plaque.

Known prior art includes various dentifrices, dentifricating paste, powders, and microbial liquids which are employed for cleaning the teeth. As a general matter, these dentrifices contain a mixture of various ingredients including such materials as polishing agents and abrasives for scouring and scrubbing the teeth, and which are further operable, to some degree, to neutralize various acids present in the gaps between the teeth. These same substances further inhibit, to some extent, the subsequent growth of various forms of bacteria that contribute to the development of caries and other disorders. While the prior art compositions have varying degrees of success, these compositions have not been particularly useful in arresting decalcification and other systemic diseases which are exasperated by the use of braces.

Brushing with current toothpaste's and rinses with high fluoride concentrations are not able to curb the acid production of carbohydrates that feed the dental plaque resulting in high decay rates and periodontal disease unless the teeth are cleaned properly. It is a well accepted fact that dental plaque when allowed to accumulate on tooth surfaces can eventually lead to gingivitis, periodontal disease, caries and calculus. Thus, it is apparent that effective removal of deposits of dental plaque is absolutely essential for oral health. Accordingly, a proper oral hygiene practice which may be carried out by an individual on his or her own teeth or by a dentist, necessitates readily available means of identification and location of plaque deposits in the oral cavity. Since dental plaque is usually transparent and colorless and not easily visible, an individual frequently is not aware of the quantity or the location of dental plaque present in the mouth. Therefore, it is desirable to use plaque-disclosing compositions to identify areas of the mouth where plaque buildup is a problem. The use of disclosing compositions motivate a person in the early removal of dental plaque by showing the presence and quantity of plaque.

Accordingly, dye indicators for dental plaque as a means of measuring tooth cleanliness and to effect proper oral hygiene practices, have been widely explored in the prior art. All of these adjuncts, however, are very time consuming and require tremendous effort on the part of the patient. Therefore, compliance with such a vast array of products is a constant struggle for both the orthodontic team as well as the patient/parent. This is very distressful to the patient/parent, who is very upset that they are spending so much of their hard earned money to straighten their children's teeth (or their own teeth) and ending up with teeth that need even more work due to decay. In fact, studies show that it is adult patients, as much as children and teenagers, who end up with decalcification due to poor oral hygiene.

Although there are several oral hygiene aids available to the public, there currently is no one product that combines everything together, which can easily be used by the patient, whether at home, school, or travel. What is lacking in the art is a product that combines several oral hygiene aids together, which can easily be used by the patient, whether at home, school, or travel.

### SUMMARY OF THE INVENTION

The present invention relates to oral compositions for identifying plaque located in a person's mouth comprising a dental disclosing agent composition for revealing the presence of dental plaque in a person's mouth including natural colorants derived from a red beet extract, a pomegranate concentrate, and one or more of lycopene, red beet powder extract, and purple carrot extract powder, and a dental disclosing agent delivery composition for delivering the dental disclosing agent to a person's mouth.

The present invention also relates to oral compositions for identifying plaque located in a person's mouth comprising a stannous fluoride and a disclosing agent including natural colorants derived from a red beet extract, a pomegranate concentrate, and one or more of lycopene, red beet powder extract, and purple carrot extract powder. The present invention provides a method for effective delivery of stannous-containing compositions with effective tartar control by administering to a subject a stable dentifrice composition comprising a clinically effective amount of stannous fluoride and/or other stannous salts in combination with a disclosing substance to highlight areas to be cleaned.

Accordingly, it is an object of the present invention to provide an oral composition for identifying plaque located in a person's mouth which is safe to use.

It is a further objective of the present invention to provide an oral composition for identifying plaque located in a person's mouth which prevents possible harm associated with using synthetic colorants as a source of dental disclosing agents.

It is yet another objective of the instant invention to provide an oral composition for identifying plaque located in a person's mouth which uses natural colorants derived from fruits and vegetables.

It is a still further objective of the present invention to provide an oral composition for identifying plaque located in a person's mouth which uses natural colorants derived from a red beet extract, a pomegranate concentrate, and one or more of lycopene, red beet powder extract, and purple carrot extract powder,

It is yet another objective of the present invention to provide an oral composition for identifying plaque located in a person's mouth which significantly reduces the amount of decalcification in patients.

It is still another objective of the present invention to provide an oral composition for identifying plaque located in a person's mouth which significantly reduces the amount of tooth decay in patients.

It is a further objective of the present invention to combine all of the necessary adjuncts of proper oral hygiene into one simple oral composition which can be used in one application and therefore result in a decalcification free (and therefore cavity free) mouth at the end of orthodontic treatment. The time involved for the patient is greatly reduced and this results in much greater compliance and success for the patients.

It is yet another objective of the present invention to provide an oral composition having a dental disclosing agent composition for revealing the presence of dental plaque in a person's mouth including natural colorants derived from a red beet extract, a pomegranate concentrate, and one or more of lycopene, red beet powder extract, and purple carrot extract powder, and a dental disclosing agent delivery composition for delivering the dental disclosing agent to a person's mouth that lets the patient see immediately while brushing, where there is plaque build-up on their teeth. Once the stain is removed during the brushing process, the teeth are clean and the risk of decalcification is minimized.

It is a still further objective of the present invention to provide an oral composition that combines a significant concentration of a fluoride source (which increases the strength of the tooth enamel and reduces the risk of decay), in conjunction with a disclosing agent that lets the patient see immediately while brushing, where there is plaque build-up on their teeth. Once the stain is removed during the brushing process, the teeth are clean and the risk of decalcification is minimized.

It is a still further objective of the present invention to provide an oral composition that combines a significant concentration of stannous fluoride (which increases the strength of the tooth enamel and reduces the risk of decay), in conjunction with a disclosing agent that lets the patient see immediately while brushing, where there is plaque build-up on their teeth. Once the stain is removed during the brushing process, the teeth are clean and the risk of decalcification is minimized.

It is yet another objective of the invention to provide an oral composition that allows a patient to visually determine the amount of brushing necessary in view of the dental abrasive employed to maximize cleaning and minimizing dentin abrasion.

These and other features, aspects, and advantages of the present invention will become evident to those skilled in the art from the detailed description which follows.

### DETAILED DESCRIPTION OF THE PREFERRED EMBODIMENT

This invention is an oral composition, in the form of a toothpaste, that combines a dental disclosing agent composition for revealing the presence of dental plaque in a person's mouth. The oral composition includes natural colorants derived from a red beet extract, a pomegranate concentrate, and one or more of lycopene, red beet powder extract, and purple carrot extract powder, and a dental disclosing agent delivery composition for delivering the dental disclosing agent to a person's mouth that lets the patient see immediately while brushing, where there is plaque build-up on their teeth. Once the stain is removed during the brushing process, the teeth are clean and the risk of decalcification is minimized. This product will replace any other toothpaste the patient has been using, and is excellent for the entire family to use daily.

Disclosing agents are known in the art and include the use various types of dyes. A natural red dye from sugar beets is disclosed in U.S. Pat. No. 4,431,628. U.S. Pat. No. 7,182,935 disclosures a plaque evidencing composition using natural colorants extracted from acai and urucum. Organic dyes such as erythrosin as disclosed in U.S. Pat. No. 3,309,274 by Brilliant which utilizes the fluorescent synthetic dyes, FDC colors Red #3, Green #8, Red #19, Red #22, Red #28, Yellow #7 and Yellow #8, which are invisible to the naked eye under normal daylight or artificial light, and only becomes visible by using light of the proper wave length. When appropriately filtered light strikes the fluorescent dye, any tartar, calculus, decay, etc. will glow brightly in its respective color. U.S. Pat. No. 3,624,219 by Perlitsch employs erythrosin as it persists in the mouth to the degree desirable for plaque-disclosing purposes. Block patents U.S. Pat. Nos. 3,723,613 and 4,064,229 developed a two-tone dye test comprising the combination of the FDC Red #3 (erythrosin) with either FDC Green #3, FDC Blue #1 or Hercules Green Shade 3 in order to obtain differential staining, i.e. thick old plaques stain blue and thin new plaques stain red.

Some synthetic dyes or artificial colorants have been linked to numerous diseases or illnesses, including asthma, thyroid tumors, depression and anxiety, and attention deficient disorders, particularly when used by children. In addition, several synthetic dyes or artificial colorants are thought to be carcinogenic. In fact, several European countries prohibit the use of several synthetic dyes or artificial colorants. While the research as to the harmful effects for some of these dyes may not be conclusive, the instant invention overcomes the risk of possible harmful effects by providing an oral composition which utilizes natural colorants.

As used herein, the term “natural colorants” describes colorants, such as lakes, dyes, chemicals, including but not limited to phytochemicals, or pigments, derived from or extracted from plants, algae, spices, herbs, or food sources, including but not limited to fruits and vegetables.

Accordingly, a dental disclosing agent used in the instant invention must be capable of adequately penetrating the plaque deposit and stain the plaque so as to be readily visible to the user, without producing an excessively prolonged staining effect. This staining efficacy must be selective so as to identify the areas of plaque-formation on all tooth surfaces and not stain gingival or other oral tissues. This selective staining efficacy must be coupled with easy removability from the mouth by simply brushing, washing, or rinsing after use. In addition, the taste must be pleasant and acceptable to the user, and the color must be pleasing. Lastly, it must be harmless and non-toxic.

The oral composition of the present invention includes a dental disclosing agent delivery composition which is made up of various ingredients, both active and inactive ingredients, which are capable of being mixed together in the form of a toothpaste for delivering the dental disclosing agent to the oral cavity and for providing various teeth cleaning and maintenance functionality. While the oral composition is preferably formulated as a toothpaste, other means of delivery, such as gels or liquids can be formulated.

In some embodiments, the dental disclosing agent delivery composition includes surfactants. Suitable non-limiting representative surfactants may include sulfated butyl oleate, medium and long chain fatty acid esters, sodium oleate, salts of fumaric acid, potassium glomate, organic acid esters of mono- and diglycerides, stearyl monoglyceridyl citrate, succistearin, dioctyl sodium sulfosuccinate, glycerol tristearate, lecithin, hydroxylated lecithin, sodium lauryl sulphate, sodium dodecyl sulphate, ammonium lauryl sulfae, acetylated monoglycerides, succinylated monoglycerides, monoglyceride citrate, ethoxylated mono- and diglycerides, sorbitan monostearate, calcium stearyl-2-lactylate, sodium stearyl lactylate, lactylated fatty acid esters of glycerol and propylene glycerol, glycerol-lactoesters of C.sub.8-C.sub.24 fatty acids, polyglycerol esters of C.sub.8-C.sub.24 fatty acids, propylene glycol alginate, sucrose C.sub.8-C.sub.24 fatty acid esters, diacetyl tartaric and citric acid esters of mono- and diglycerides, triacetin, sarcosinate surfactants, isethionate surfactants, tautate surfactants, pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures thereof.

In some embodiments, the dental disclosing agent delivery composition includes a fluoride source as fluoride is known to prevent tooth decay and makes teeth stronger by incorporating itself into teeth enamel. Fluoride compounds have found widespread usage as effective ingredients for inhibiting dental caries. Among those fluoride compounds, fluoride salts which contain stannous ions (e.g. stannous fluoride) have been reported to cause an increase in the fluoride uptake by the dental enamel and consequently in acid-resistance of the enamel after treatment as compared with fluoride salts which do not contain stannous ions. U.S. Pat. No. 3,105,798 discloses a dentifrice composition consisting essentially of a water-soluble fluoride salt, stannous tin and a water-soluble source of six carbon aldonate groups capable of forming water-soluble complexes with stannous tin, the molar ratio of the aldonate group to stannous tin being in the range of from about one:one to about three:one, the molar ratio of stannous tin to fluoride ions being greater than one:one, and the dentifrice having a pH of from about 5 to 7. Crystals of sodium pentafluorostannite (NaSn.sub.2 F.sub.5) obtained by reacting one mole of sodium fluoride with two moles of stannous fluoride are described in U.S. Pat. No. 3,490,866. The use of stannous salts of polyphosphonic acids such as methanediphosphonic acid or ethane-1-hydroxy-1,1-diphosphonic acid described in U.S. Pat. No. 3,549,677. The prior art compositions containing stannous ions and fluoride ions are effective to some extent for dental caries reduction, but their effectiveness is not so extremely high. Moreover, they require repeated application because of their lower reactivity to the tooth surface upon application or readily decreased retention of effectiveness.

Suitable non-limiting representative forms of fluoride include sodium monofluorophosphate, sodium fluoride, and stannous fluoride. Stannous fluoride is commonly incorporated into toothpastes for therapeutic efficacy in the control of dental caries. Stannous fluoride gels, rinses, and dentifrices have since been shown to provide clinical efficacy for the reduction of dental caries, dentinal hypersensitivity, dental plaque and gingivitis. In addition to these clinical effects, formulations containing stannous fluoride may also help to Provide improved breath benefits through chemical and antibacterial actions.

In some embodiments, the dental disclosing agent delivery composition includes abrasives. Suitable non-limiting representative abrasives include silicas, aluminas, phosphates, carbonates and combinations thereof. In some embodiments, the abrasive agent is a silica selected from: precipitated silica, silica gels and combinations thereof. Moreover, in some embodiments the abrasive agent is selected from the following: calcium carbonate, sodium bicarbonate, sodium metaphosphate, potassium metaphosphate, tricalcium phosphate, dicalcium phosphate, dehydrated dicalcium phosphate, calcium hydrogen orthophosphate, and combinations thereof.

Hydrated silica is used as a dental abrasive to maximize cleaning and minimizing tooth abrasion. The ability to optimize such characteristics in the past has been limited generally to controlling the structures of the individual components utilized for such purposes for there has been no way of determining how long an individual should be brushing. For instance, if the teeth are clean then the use of a dental abrasive is of no effect toward the cleaning and may actually be harmful. However, if the teeth are unclean then the use of a dental abrasive is necessary to effectively remove the foreign particles. Prior art was limited to improving the dental abrasive and no teaching was directed to determining how much abrasion contact was necessary. The instant invention allows the consumer to visually determine the amount of brushing required, namely the brushing must be continued until all disclosing material is removed. Examples of modifications in precipitated silica structures for such dentifrice purposes are described in the art within such publications as U.S. Pat. Nos. 3,967,563, 3,988,162, 4,420,312, and 4,122,161 to Wason, U.S. Pat. Nos. 4,992,251 and 5,035,879 to Aldcroft et al, U.S. Pat. No. 5,098,695 to Newton et al, and U.S. Pat. Nos. 5,891,421 and 5,419,888 to McGill et al. Modifications in silica gels have also been described within such publications as U.S. Pat. No. 5,647,903 to McGill et al, U.S. Pat. No. 4,303,641, to DeWolf, II et al, U.S. Pat. No. 4,153,680, to Seybert, and U.S. Pat. No. 3,538,230, to Pader et al. Such disclosures teach improvement in such silica materials in order to impart increased cleaning capacity and reductions in dentin abrasion levels for dentifrice benefits. However, these improvements lack the ability to deliver preferred property levels that accord a dentifrice producer the ability incorporate such an individual material in different amounts with disclosure components in order to effectuate different resultant levels of such cleaning and abrasion characteristics. Silica combinations involving compositions of differing particle sizes and specific surface areas are disclosed in U.S. Pat. No. 3,577,521. to Karlheinz Scheller et al, U.S. Pat. No. 4,618,488 to Macyarea et al, U.S. Pat. No. 5,124,143 to Muhlemann, and U.S. Pat. No. 4,632,826 to Ploger et al. Such resultant dentifrices, however, fail to provide desired levels of abrasion and cleaning simultaneously by use of a visual indicator. The instant invention combines the use of a dental disclosing agent, a fluoride ion, and a dental abrasive within a single paste.

In some embodiments, the dental disclosing agent delivery composition includes humectants such as, but not limited to, water, sorbitol, glycerin, xylitol, or combinations thereof.

In some embodiments, the dental disclosing agent delivery composition includes thickeners. Suitable non-limiting representative thickeners include, methyl cellulose, alginates, carrageenan, xanthan gum, gelatin, carob, tragacanth, locust bean, and carboxymethyl cellulose, cellulose gum, acidulants such as malic acid, adipic acid, citric acid, tartaric acid, fumaric acid, and mixtures thereof.

In some embodiments, the dental disclosing agent delivery composition includes preservatives. Suitable non-limiting representative preservatives include sodium benzoate, ethyl paraben, methyl paraben, and combinations thereof.

In some embodiments, the dental disclosing agent delivery composition includes flavoring agents. Suitable non-limiting representative flavorings include, natural and artificial flavors, such as synthetic flavor oils and flavoring aromatics and/or oils, oleoresins and extracts derived from plants, leaves, flowers, fruits, and so forth, and combinations thereof. Non-limiting representative flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including lemon, orange, lime, grapefruit, and fruit essences including apple, pear, peach, grape, strawberry, raspberry, cherry, plum, pineapple, apricot, aldehyde flavorings, and combinations thereof.

In some embodiments, the dental disclosing agent delivery composition includes sweeteners. Suitable non-limiting representative sweeteners include water-soluble sweeteners, water-soluble artificial sweeteners, water-soluble sweeteners derived from naturally occurring water-soluble sweeteners, dipeptide based sweeteners, and protein based sweeteners, including mixtures thereof. Illustrative examples include soluble saccharin salts, i.e., sodium or calcium saccharin salts, ihydrochalcones, monellin, steviosides, glycyrrhizin, dihydroflavenol, and sugar alcohols such as sorbitol, mannitol, maltitol, and L-aminodicarboxylic acid aminoalkenoic acid ester amides

In some embodiments, the dental disclosing agent delivery composition includes coloring agents for providing a desired color of the oral composition itself. Non-limiting representative coloring agents may include titanium dioxide, food colorings and dyes, such as F.D.&C dyes and lakes. Preferably, the coloring agents are naturally derived.

In some embodiments, the dental disclosing agent delivery composition includes vitamins, such as Vitamin E, alpha-tocopherol.

In some embodiments, the dental disclosing agent delivery composition includes antimicrobial agents/antibacterial agents. Suitable non-limiting representative antimicrobial agents/antibacterial agents include triclosan, xylitol, or cetylpyridium chloride employed alone or in combination thereof. The antibacterial agent extends the shelf life of the dental composition but can further minimize the microbial population in the mouth. Since the oral environment is conducive to microbial growth and subject to the reintroduction of food and microorganisms, and because plaque and calculus are continually being deposited on teeth, the composition must address the microbial growth during the cleaning process. Dental plaque which forms on tooth surfaces and restorations are colonies of harmful bacteria, which cannot be flushed away by simply rinsing with water. Active brushing of the teeth is required to remove the adherent plaque and the use of triclosan will destroy the harmful bacteria.

In some embodiments, the dental disclosing agent delivery composition includes chelating agents. Non-limiting chelating agents include pyrophosphates, triphosphates, polyphosphates, polyphosphonates, dialkali metal pyrophosphate salt, a tetra alkali, tetrasodium pyrophosphate, tetrapotassium pyrophosphate, sodium tripolyphosphate, polyphosphate salt, EDTA (ethylenediaminetetraacetate) and salts of EDTA such as calcium disodium ethylenediaminetetraacetate dehydrate, (CaNa₂EDTA.2H₂O, Calcium Disodium EDTA FCC) or disodium ethylenediaminetetraacetate dehydrate (Na₂H₂EDTA.2H₂O, Disodium EDTA FCC, Edetate Disodium USP), and combinations thereof. The use of EDTA, and its salts, either in the dental disclosing agent delivery composition or disclosing agent composition serves several functions. First, it will act as a plaque softening and degrading agent, aiding in the removal of plaque. The actual process would involve chelation of trace metals having multivalent ions, such as iron (Fe), copper (Cu), manganese (Mn), calcium (Ca), magnesium (Mg), or zinc (Zn). In addition, EDTA, and its salts, by chemically binding and effectively chelating the trace metals, minimizes the effects the trace metals have on the color, flavor, and shelf-life capacity.

In some embodiments, the dental disclosing agent delivery composition includes anti-tartar agents such as tetrasodium pyrophosphate.

The following tables are illustrative embodiments of the dental disclosing agent delivery composition and the dental disclosing agent composition. While Table 1 describes the dental disclosing agent delivery composition in the preferred embodied form of a toothpaste, the disclosing agent delivery composition may be formulated in other forms, such as, but not limited to, gel or liquid formulations.

TABLE 1
Dental disclosing agent delivery composition:

| | Quantity % |
|---|---|
| Component | w/w or w/v |
| Dicalcium phosphate | 37.0-45.0% |
| (calcium hydrogen orthophosphate) | |
| Water | 5.0-25.0% |
| Sorbitol (D-Glucitol) | 10.0-20.0% |
| Glycerin | 8.0-20.0% |
| Tetrasodium pyrophosphate (TSPP) | 0.25-3.0% |
| Sodium lauryl sulphate | 0.5-2.0% |
| (sodium dodecyl sulphate) | |
| Sodium saccharin | 0.10-2.5% |
| (1,2-Benzisothiazol-3(2H)- | |
| one,1,1-dioxide, sodium salt) | |
| Titanium dioxide | 0.0-1.5% |
| Dental disclosing agent composition | As needed |
| Sodium benzoate | 0.0-3.0% |
| Cellulose gum (Carboxymethyl cellulose) | 0.5-4.0% |
| Alpha-tocopherol (Vitamin-E Natural) | 0.0-0.5% |
| Sodium Fluoride (Active ingredient) | 0.1-0.25% |
| Triclosan (2,4,4′-Trichloro- | 0.0-3.0% |
| 2′hydroxydiphenyl ether) | |
| calcium disodium ethylenediaminetetra- | 0.0-1.5% |
| acetate dehydrate (CaNa₂EDTA•2H₂0, | |
| Calcium Disodium EDTA FCC) | |
| disodium ethylenediaminetetraacetate | 0.0-1.5% |
| dehydrate (Na₂H₂EDTA•2H₂0, Disodium | |
| EDTA FCC, Edetate Disodium USP) | |

Tables 2-4 illustrate several embodiments of the dental disclosing agent composition in accordance with the instant invention.

TABLE 2
Dental disclosing agent composition, Example 1:

| | Quantity % |
|---|---|
| Component | w/w or w/v |
| Water | 10.0-40.0% |
| Sodium lauryl sulphate | 0.0-10% |
| (sodium dodecyl sulphate) | |
| calcium disodium ethylenediaminetetra- | 0.0-1.62% |
| acetate dehydrate (CaNa₂EDTA•2H₂0, | |
| Calcium Disodium EDTA FCC) | |
| disodium ethylenediaminetetraacetate | 0.0-1.62% |
| dehydrate (Na₂H₂EDTA•2H₂0, Disodium | |
| EDTA FCC, Edetate Disodium USP) | |
| Sodium Benzoate | 0.0-1.30% |
| Red Beet Extract (extracted from red | 10.0-40.0% |
| beetroot, concentrated and pasteurized | |
| by physical means to standardized color | |
| Lycopene (Extracted from tomatoes, | 5.0-30.0% |
| emulsified and stabilized) | |
| Pomegranate Concentrate (extracted from | 0.0-8.0% |
| pomegranates, concentrated and pasteurized | |
| and/or stabilized by physical means) | |

In addition to extracting lycopene from tomatoes, other sources, such as red carrots, watermelons, papayas, or plants and algae may be used as well.

TABLE 3
Dental disclosing agent composition, Example 2

| | Quantity % |
|---|---|
| Component | w/w or w/v |
| Water | 25.0-50.0% |
| Sodium lauryl sulphate | 0.0-10.0% |
| (sodium dodecyl sulphate) | |
| calcium disodium ethylenediaminetetra- | 0.0-1.73% |
| acetate dehydrate (CaNa₂EDTA•2H₂0, | |
| Calcium Disodium EDTA FCC) | |
| disodium ethylenediaminetetraacetate | 0.0-1.73% |
| dehydrate (Na₂H₂EDTA•2H₂0, Disodium | |
| EDTA FCC, Edetate Disodium USP) | |
| Sodium Benzoate | 0.0-1.38% |
| Red Beet Extract (extracted from red | 5.0-35.0% |
| beetroot, concentrated and pasteurized | |
| by physical means to standardized color | |
| Red Beet Extract Powder | 5.0-35.0% |
| (extracted from red beetroot, mixed with | |
| maltodextrin and spray dried)) | |
| Pomegranate Concentrate (extracted from | 0.0-15.0% |
| pomegranates, concentrated and pasteurized | |
| and/or stabilized by physical means) | |

TABLE 4
Dental disclosing agent composition, Example 3:

| | Quantity % |
|---|---|
| Component | w/w or w/v |
| Water | 10.0-70.0% |
| Sodium lauryl sulphate | 0.0-10% |
| (sodium dodecyl sulphate) | |
| calcium disodium ethylenediaminetetra- | 0.0-1.73% |
| acetate dehydrate (CaNa₂EDTA•2H₂0, | |
| Calcium Disodium EDTA FCC) | |
| disodium ethylenediaminetetraacetate | 0.0-1.73% |
| dehydrate (Na₂H₂EDTA•2H₂0, Disodium | |
| EDTA FCC, Edetate Disodium USP) | |
| Sodium Benzoate | 0.0-2.0% |
| Red Beet Extract (extracted from red | 0.0-10.0% |
| beetroot, concentrated and pasteurized | |
| by physical means to standardized color | |
| Red Beet Extract Powder | |
| (extracted from red beetroot, mixed with | 0.0-10.0% |
| maltodextrin and spray dried)) | |
| Purple Carrot Extract Powder (Extracted from | 15.0-85.0% |
| carrots, clarified, concentrated by physical | |
| means and spray dried) | |
| Pomegranate Concentrate (extracted from | 0.0-15.0% |
| pomegranates, concentrated and pasteurized | |
| and/or stabilized by physical means) | |

The process for producing the dental disclosing agent delivery composition of the oral composition for identifying plaque located in a person's mouth in accordance with the instant invention is a four phase process:

Phase A: The process begins by mixing the glycerin, sorbitol, and cellulose gum. The components are combined using moderate agitation until the liquid is clear and has no lumps.

Phase B: Tetrasodium pyrophosphate, sodium saccharin, sodium fluoride, and sodium benzoate is added to water (distilled or RO/DI) at a temperature of 50-60 degrees Celsius. The mixture is mixed with moderate agitation until the components are dissolved (Phase B).

Phase B component is added to Phase A component using elevated agitation. The two components are mixed until clear (no lumps).

Phase C: Dicalcium phosphate is added to the Phase A/Phase B mixture using elevated agitation. The Mixture is agitated until uniform. The mixture is then slowly and completely de-aerated.

Phase D: Sodium lauryl sulphate is added to the dental disclosing agent composition, and if used flavoring. The components are mixed using moderate agitation until the mixture is uniform.

The dental disclosing agent composition is produced using a two phase process:

Phase 1: Sodium lauryl sulphate, EDTA, and sodium benzoate is added to water at a temperature of 50-60 degrees Celsius. The mixture is mixed until clear.

Phase 2: Desired extracts are added to the Phase 1 mixture and allowed to mix using low agitation. If using a mixture of powder extracts and liquid extracts, powder extracts should be added first. The mixture is allowed to mix until it is clear. No aeration is performed. This mixture is then ready to be added to the dental disclosing agent delivery composition at prescribed quantities at the finished temperature.

It is to be understood that while a certain form of the invention is disclosed, it is not to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. An oral composition for identifying plaque located in a person's mouth comprising:
a dental disclosing agent composition for revealing the presence of dental plaque in a person's mouth comprising natural colorants derived from a red beet extract, a pomegranate concentrate, and one or more of lycopene, red beet powder extract, and purple carrot extract powder; and
a dental disclosing agent delivery composition for delivering said dental disclosing agent to a person's mouth.

2. The oral composition for identifying plaque located in a person's mouth according to claim 1 wherein said dental disclosing agent delivery composition further comprises one or more of surfactants, a fluoride source, abrasives, humectants, thickeners, preservatives, antimicrobial agents, flavoring agents, sweeteners, vitamins, and coloring agents.

3. The oral composition for identifying plaque located in a person's mouth according to claim 2 wherein said fluoride source is sodium fluoride.

4. The oral composition for identifying plaque located in a person's mouth according to claim 2 wherein said anti-microbial agent is triclosan.

5. The oral composition for identifying plaque located in a person's mouth according to claim 2 further including flavorings.

6. The oral composition for identifying plaque located in a person's mouth according to claim 5 wherein said flavorings are derived from natural sources.

7. The oral composition for identifying plaque located in a person's mouth according to claim 1 wherein said dental disclosing agent composition includes red beet extract, lycopene, and pomegranate concentrate.

8. The oral composition for identifying plaque located in a person's mouth according to claim 7 wherein said dental disclosing agent delivery composition further comprises one or more of surfactants, a fluoride source, abrasives, humectants, thickeners, preservatives, antimicrobial agents, flavoring agents, sweeteners, vitamins, and coloring agents.

9. The oral composition for identifying plaque located in a person's mouth according to claim 7 wherein said fluoride source is sodium fluoride.

10. The oral composition for identifying plaque located in a person's mouth according to claim 7 wherein said anti-microbial agent is triclosan.

11. The oral composition for identifying plaque located in a person's mouth according to claim 7 wherein said dental disclosing agent composition further includes at least one chelating agent.

12. The oral composition for identifying plaque located in a person's mouth according to claim 7 wherein said at least one chelating agent includes calcium disodium ethylenediaminetetraacetate dehydrate and disodium ethylenediaminetetraacetate dehydrate.

13. The oral composition for identifying plaque located in a person's mouth according to claim 7 further including flavorings.

14. The oral composition for identifying plaque located in a person's mouth according to claim 7 wherein said flavorings are derived from natural sources.

15. The oral composition for identifying plaque located in a person's mouth according to claim 1 wherein said dental disclosing agent composition includes red beet extract, red beet extract powder, and pomegranate concentrate.

16. The oral composition for identifying plaque located in a person's mouth according to claim 15 wherein said dental disclosing agent delivery composition further comprises one or more of surfactants, a fluoride source, abrasives, humectants, thickeners, preservatives, antimicrobial agents, flavoring agents, sweeteners, vitamins, and coloring agents.

17. The oral composition for identifying plaque located in a person's mouth according to claim 16 wherein said fluoride source is sodium fluoride.

18. The oral composition for identifying plaque located in a person's mouth according to claim 16 wherein said anti-microbial agent is triclosan.

19. The oral composition for identifying plaque located in a person's mouth according to claim 16 wherein said dental disclosing agent composition further includes at least one chelating agent.

20. The oral composition for identifying plaque located in a person's mouth according to claim 19 wherein said at least one chelating agent includes calcium disodium ethylenediaminetetraacetate dehydrate and disodium ethylenediaminetetraacetate dehydrate.

21. The oral composition for identifying plaque located in a person's mouth according to claim 16 further including flavorings.

22. The oral composition for identifying plaque located in a person's mouth according to claim 21 wherein said flavorings are derived from natural sources.

23. The oral composition for identifying plaque located in a person's mouth according to claim 1 wherein said dental disclosing agent composition includes red beet extract, red beet extract powder, purple carrot extract powder, and pomegranate concentrate.

24. The oral composition for identifying plaque located in a person's mouth according to claim 23 wherein said dental disclosing agent delivery composition further comprises one or more of surfactants, a fluoride source, abrasives, humectants, thickeners, preservatives, antimicrobial agents, flavoring agents, sweeteners, vitamins, and coloring agents.

25. The oral composition for identifying plaque located in a person's mouth according to claim 24 wherein said fluoride source is sodium fluoride.

26. The oral composition for identifying plaque located in a person's mouth according to claim 24 wherein said anti-microbial agent is triclosan.

27. The oral composition for identifying plaque located in a person's mouth according to claim 24 wherein said dental disclosing agent composition further includes at least one chelating agent.

28. The oral composition for identifying plaque located in a person's mouth according to claim 27 wherein said at least one chelating agent includes calcium disodium ethylenediaminetetraacetate dehydrate and disodium ethylenediaminetetraacetate dehydrate.

29. The oral composition for identifying plaque located in a person's mouth according to claim 24 further including flavorings.

30. The oral composition for identifying plaque located in a person's mouth according to claim 29 wherein said flavorings are derived from natural sources.

31. An oral composition for identifying plaque located in a person's mouth comprising:
a dental disclosing agent composition for revealing the presence of dental plaque in a person's mouth comprising a combination of natural colorants derived from a plurality of fruit or vegetable sources, said fruit or vegetable sources including a red beet extract and a pomegranate concentrate in combination with one or more of lycopene, red beet extract powder, and purple carrot extract powder.
a dental disclosing agent delivery composition for delivering said dental disclosing agent to a person's mouth, said delivery composition comprising dicalcium phosphate in a concentration of about 37.0% to about 45.0%, water in a concentration of about 5.0% to about 25.0%, sorbitol in a concentration of about 10.0% to about 20.0%, glycerin in a concentration of about 8.0% to about 20.0%, tetrasodium pyrophosphate in a concentration of about 0.25% to about 3.0%, sodium lauryl sulphate in a concentration of about 0.5% to about 2.0%, sodium saccharin in a concentration of about 0.10% to about 2.5%, titanium dioxide in a concentration of about 0.05% to about 1.5%, sodium benzoate in a concentration of about 0.05% to about 3.0%, cellulose gum in a concentration of about 0.5% to about 4.0%, alpha-tocopherol in a concentration of about 0.1% to about 0.05% to about 0.5%, sodium fluoride in a concentration of about 0.1% to about 0.25%, triclosan in a concentration of about 0.05% to about 3.0%, calcium disodium ethylenediaminetetraacetate dehydrate in a concentration of about 0.05% to about 1.5%, and disodium ethylenediaminetetraacetate dehydrate a concentration of about 0.05% to about 1.5%.

32. The oral composition for identifying plaque located in a person's mouth according to claim 31 wherein said dental disclosing agent composition comprises water in a concentration of about 10.0% to about 40.0%, sodium lauryl sulphate in a concentration of about 0.05% to about 10.0%, calcium disodium ethylenediaminetetraacetate dehydrate in a concentration of about 0.05% to about 1.62%, disodium ethylenediaminetetraacetate dehydrate in a concentration of about 0.05% to about 1.62%, sodium benzoate in a concentration of about 0.05% to about 1.3%, red beet extract in a concentration of about 10.0% to about 40.0%, lycopene in a concentration of about 5.0% to about 30.0%, and pomegranate concentrate in a concentration of about 0.05 to about 8.0%.

33. The oral composition for identifying plaque located in a person's mouth according to claim 31 wherein said dental disclosing agent composition comprises water in a concentration of about 25.0% to about 50.0%, sodium lauryl sulphate in a concentration of about 0.05% to about 10.0%, calcium disodium ethylenediaminetetraacetate dehydrate in a concentration of about 0.05% to about 1.73%, disodium ethylenediaminetetraacetate dehydrate in a concentration of about 0.05% to about 1.73%, sodium benzoate in a concentration of about 0.05% to about 1.38%, red beet extract in a concentration of about 5.0% to about 35.0%, red beet extract powder in a concentration of about 5.0% to about 35.0%, and pomegranate concentrate in a concentration of about 0.05% to about 15.0%.

34. The oral composition for identifying plaque located in a person's mouth according to claim 31 wherein said dental disclosing agent composition comprises water in a concentration of about 10.0% to about 70.0%, sodium lauryl sulphate in a concentration of about 0.05% to about 10.0%, calcium disodium ethylenediaminetetraacetate dehydrate in a concentration of about 0.05% to about 1.73%, disodium ethylenediaminetetraacetate dehydrate in a concentration of about 0.05% to about 1.73%, sodium benzoate in a concentration of about 0.05% to about 2.0%, red beet extract in a concentration of about 0.05% to about 10.0%, red beet extract powder in a concentration of about 0.05% to about 10.0%, purple carrot extract powder from a concentration of about 15.0% to about 85.0%, and pomegranate concentrate in a concentration of about 0.05 to about 15.0%.
